(19) United States
(12) Patent Application Publication
(10) Pub. No.: US 2016/0167479 A1
(43) Pub. Date: Jun. 16, 2016

(54) Vehicle Monitoring System

(71) Applicant: Noel Morin, Hilo, HI (US)

(72) Inventor: Noel Morin, Hilo, HI (US)

(21) Appl. No.: 14/965,335

(22) Filed: Dec. 10, 2015

Related U.S. Application Data
(60) Provisional application No. 62/089,896, filed on Dec. 10, 2014.

(51) Int. Cl. B60H 1/00; B60Q 5/00; G01K 13/00; G01S 19/42; G08B 21/14; G01N 33/00; B60J 1/17; B60Q 1/52

(52) U.S. Cl. CPC B60H 1/00742; B60H 1/00757; B60J 1/17; B60Q 5/005; B60Q 1/52; G01S 19/42; G08B 21/14; G01N 33/004; G01K 13/00

(57) ABSTRACT

A vehicle monitoring system configured to cause a vehicle to perform a corrective action if a dangerous vehicle interior condition is detected and an occupant is present. The vehicle monitoring system monitors the temperature and gas, such as CO2, and compares the levels of each to a threshold value. If the threshold value is exceeded, a sound sensor determines whether or not the vehicle is occupied. If the vehicle is occupied, the vehicle monitoring system causes the vehicle to perform a corrective action such as flashing the exterior lights or opening one or more windows. The vehicle monitoring system also determines the location of the vehicle via a GPS and transmits the location of the vehicle to a third party if a dangerous condition is present and the vehicle is determined to be occupied.

### CROSS REFERENCE TO RELATED APPLICATIONS

This application claims the benefit of U.S. Provisional Application No. 62/089,896 filed on Dec. 10, 2014. The above identified patent application is herein incorporated by reference in its entirety to provide continuity of disclosure.

### BACKGROUND OF THE INVENTION

The present invention relates to vehicle monitoring systems. More specifically, the present invention relates to systems for monitoring the presence of an occupant in a vehicle and causing the vehicle to perform a corrective action if a dangerous condition in the vehicle is determined to exist.

Every year, many children are injured or even killed as a result of being in a vehicle for prolonged periods of time. Pets may also unfortunately be forgotten or purposely left in a vehicle and may suffer the same fate. Temperatures in the interior of a vehicle can quickly reach life threatening levels and can cause injury or even death to occupants. In warm temperatures, the interior of a vehicle heats up very quickly and poses great risk to living beings left inside. Likewise, a person or pet left in a vehicle in cold conditions are in danger of hypothermia or even death. Further, the concentration of carbon dioxide (CO2) increases in closed space over time as an individual continuously exhales. Increased concentrations of CO2 can lead to dizziness, fatigue, and in some cases even death from suffocation. Children may enter a vehicle by themselves and become trapped, or a parent or other adult may forget that a child is in the vehicle. Likewise, pets may become trapped in a vehicle or accidentally left in the vehicle by an individual. Therefore, there is a need for a vehicle monitoring system that provides for the correction or alleviation of dangerous vehicle conditions in order to prevent accidental injury or death to vehicle occupants such as children, pets, or others.

Some previous methods of solving the problem of accidental vehicle occupant deaths due to overheating or suffocation are ineffective because they only focus on alerting an individual of the presence of a vehicle occupant. For example, one way of determining that a vehicle is occupied is to place an individual's personal property that they carry with them, such as a purse or wallet, in the backseat. Thus, the individual will see the occupant before leaving the vehicle when the personal item is retrieved. While this helps to alleviate danger from occupants being left in a vehicle, these devices do not address instances where an individual, such as a child, enters a vehicle unsupervised and becomes stuck inside. Therefore, there is a need to provide a vehicle monitoring system that automatically corrects dangerous conditions within a vehicle when the presence of an occupant is detected.

### SUMMARY OF THE INVENTION

In view of the foregoing disadvantages inherent in the known types of vehicle monitoring system now present in the prior art, the present invention provides a vehicle monitoring system wherein the same can be utilized for providing convenience for the user when monitoring a vehicle for unsafe conditions and causing a vehicle to perform a corrective action if an occupant is determined to be present and subjected to the unsafe condition. The present system comprises a vehicle monitoring system in operative and electrical communications with the electrical systems of a vehicle, wherein the vehicle monitor causes the vehicle to perform corrective actions such as lowering windows, turning on air condition, flashing exterior lights, or sounding a horn, upon the determination of the existence of an unsafe condition such as an unsafe temperature or unsafe CO2 concentration.

One object of the present invention is to provide a vehicle monitoring device having all of the advantages but none of the disadvantages found in the prior art.

Another object of the present invention is to provide a vehicle monitoring system that causes corrective measures to be taken by the vehicle.

A further object of the present invention is to provide a vehicle monitoring system having a GPS, wherein the vehicle monitoring system sends information to a third party upon the existence of a dangerous condition and an occupant subjected thereto, the information including the geographic location of the vehicle monitoring device as determined by the GPS.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a detail view of a vehicle monitoring system according to the present invention being used in a vehicle.

FIG. 2 shows a schematic view of the components of a vehicle monitoring system of the present invention.

FIG. 3 shows a flowchart detailing the steps of a method performed by a vehicle monitoring system according to present invention.

FIG. 4 shows a schematic view of a vehicle monitoring system according to the present invention transmitting a signal to a third party.

### DETAILED DESCRIPTION OF THE INVENTION

Reference is made herein to the attached drawings. Like reference numerals are used throughout the drawings to depict like or similar elements of the vehicle monitoring system. For the purposes of presenting a brief and clear description of the present invention, the preferred embodiment will be discussed as used for monitoring a vehicle for occupants and taking a corrective action if a dangerous condition is detected. The figures are intended for representative purposes only and should not be considered to be limiting in any respect.

As used herein, “logic” refers to (i) logic implemented as computer instructions and/or data within one or more computer processes and/or (ii) logic implemented in electronic circuitry.

As used herein, “computer-readable medium” excludes any transitory signals, but includes any non-transitory data storage circuitry, e.g., buffers, cache, and queues, within transceivers of transitory signals.

As used herein, “occupant” refers to any living thing, human or animal, that is present within the interior of a vehicle.

According to some embodiments, the operations, techniques, and/or components described herein can be implemented by an electronic device, which can include one or more special-purpose computing devices such as a vehicle monitoring system. The special-purpose computing devices can be hard-wired to perform the operations, techniques, and/or components described herein, or can include digital electronic devices such as one or more application-specific integrated circuits (ASICs) or field programmable gate arrays (FPGAs) that are persistently programmed to perform the operations, techniques and/or components described herein, or can include one or more general purpose hardware processors programmed to perform such features of the present disclosure pursuant to program instructions in firmware, memory, other storage, or a combination. Such special-purpose computing devices can also combine custom hard-wired logic, ASICs, or FPGAs with custom programming to accomplish the technique and other features of the present disclosure. The special-purpose computing devices can be desktop computer systems, portable computer systems, handheld devices, networking devices, or any other device that incorporates hard- wired and/or program logic to implement the techniques and other features of the present disclosure.

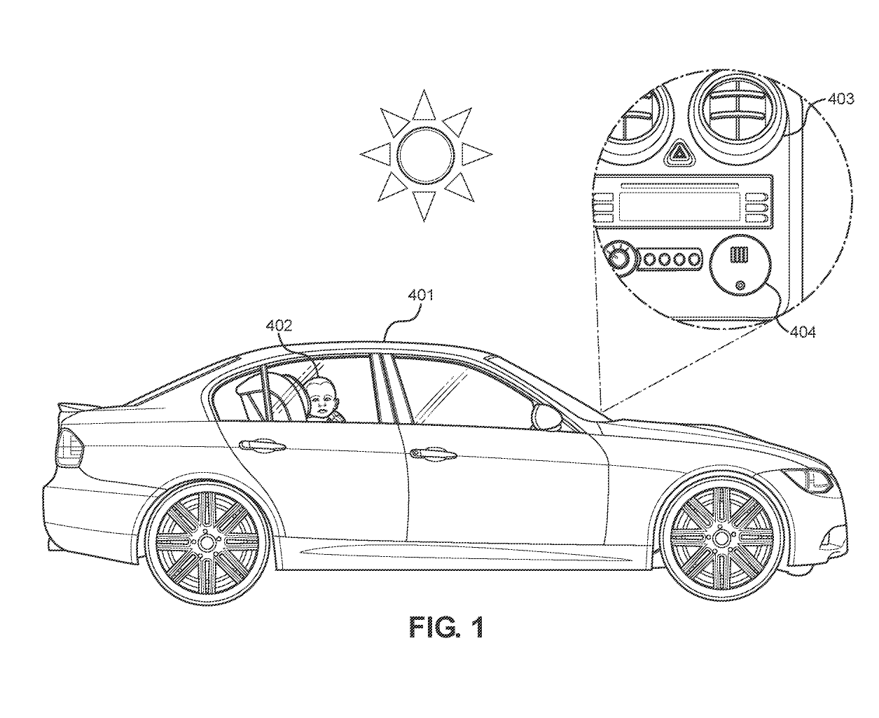

Referring now to FIG. 1, there is shown a detail view of a vehicle monitoring system according to the present invention being used in a vehicle. The vehicle monitor 404 is used in conjunction with a vehicle 401. The vehicle monitor 404 utilizes a variety of sensors to detect for the presence of an occupant 402 in a vehicle 401 and any unsafe conditions therein. If unsafe conditions are detected within the vehicle 401, the vehicle monitor 404 communicates with the vehicle systems in order to take corrective actions. The vehicle monitor 404 is in electrical and operable communication with the systems of the vehicle 401, such as the air conditioning, window controls, and exterior lights, via the vehicle circuitry. The vehicle monitor 404 comprises a connection that is configured to plug in to a port on the vehicle dashboard 403 in order to connect the vehicle monitor thereto. In an alternate embodiment, the vehicle monitor 404 may be affixed to another portion of the vehicle and connected to the vehicle electrical systems via one or more wires. The vehicle monitor 404 may also be attached to a child's car seat or may be non-removably integrated into the vehicle.

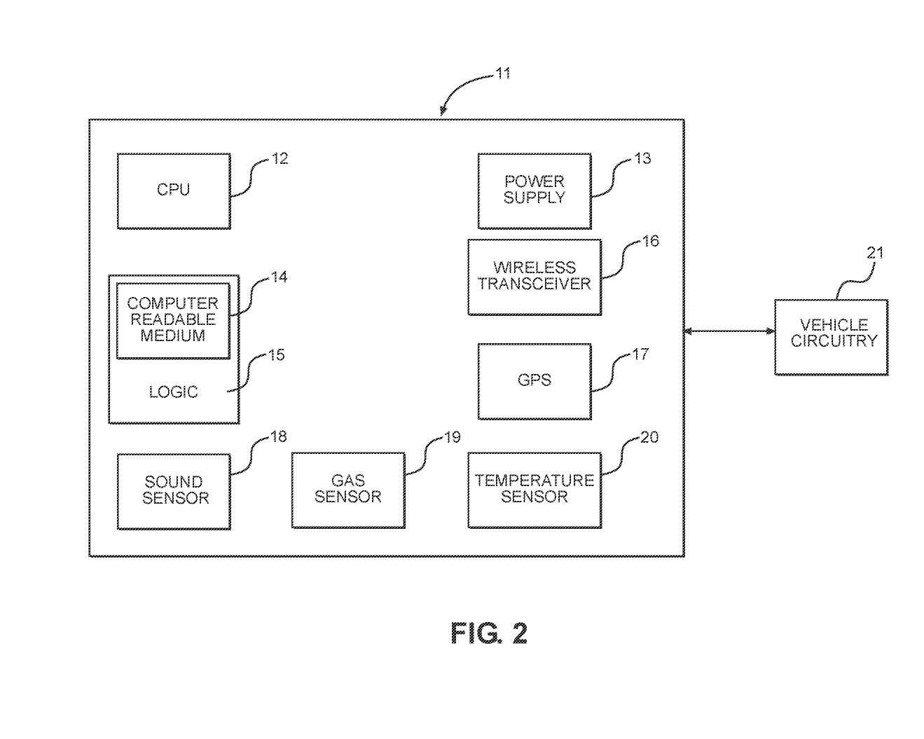

Referring now to FIG. 2, there is shown a schematic view of the components of a vehicle monitor according to the present invention. The vehicle monitor 11 is in operable and electrical communication with a vehicle's internal circuitry 21 such that the vehicle monitor 11 causes the vehicle to perform a corrective action upon the existence of a dangerous condition such as, for example, a child left in a hot vehicle. The vehicle monitor 11 comprises a power supply 13 electrically and operatively connected to a computer processing unit (CPU) 12, a computer readable medium 14 such as a memory, a wireless transceiver 16, a GPS 17, and a variety of sensors such as a sound sensor 18, gas sensor 19, and temperature sensor 20. The power supply 13 may be an internal battery or the vehicle monitor 11 may draw power from the vehicle battery. The power supply 13 may further comprise reference battery back-ups in order to ensure that the device remains powered in the event of a disconnect or primary power source interruption.

The CPU 12 is configured to execute a logic 15 stored on the computer readable medium 14. The logic 15 causes the vehicle monitoring system to perform a method wherein the vehicle performs a corrective action upon the detection of one or more dangerous conditions within the vehicle. The GPS 17 is utilized to determine the geographic location of the vehicle in which the vehicle monitor 11 is located. A wireless transceiver 16 is utilized to provide the geographic location of the vehicle via a transmission over a network such as a cellular network or Wi-Fi network upon the occurrence of a dangerous condition within the vehicle.

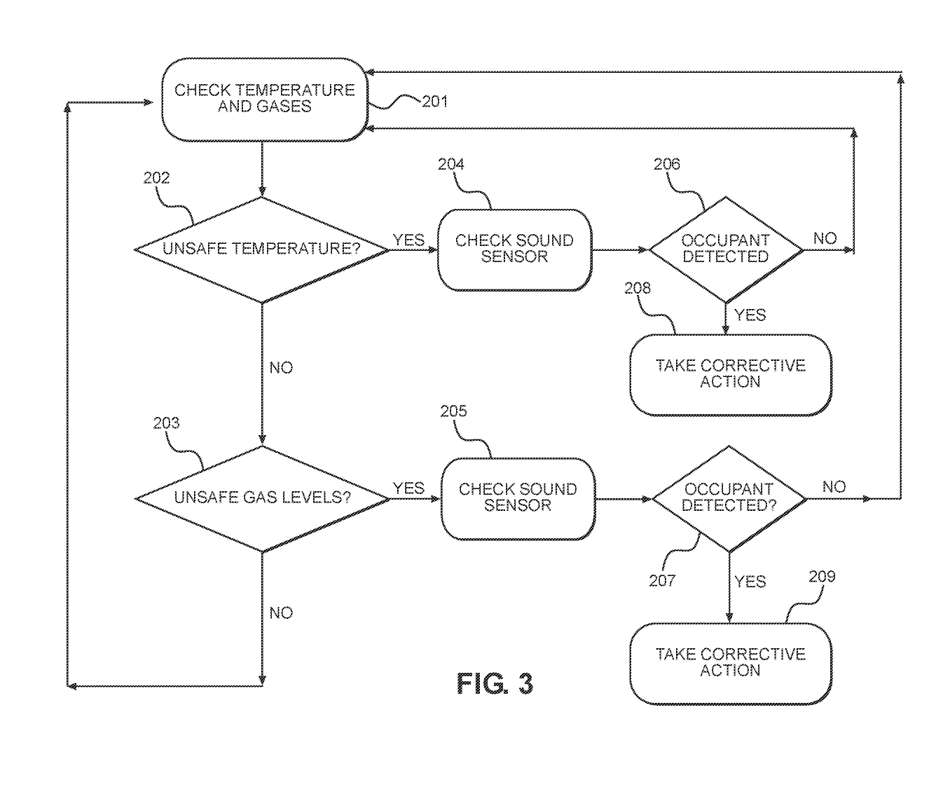

Referring now to FIG. 3, there is shown a flowchart detailing the steps of a method performed by a vehicle monitoring system according to present invention. When executed by the CPU, the logic causes the vehicle monitoring system to measure 201 the temperature and gas levels of the interior of the vehicle via the temperature and gas sensors, respectively. The gas sensor is preferably configured to detect the level of CO2 present in the interior of the vehicle, but may also be configured to detect the presence and/or concentration of other gases.

In monitoring the interior of the vehicle, the logic causes the vehicle monitoring system to determine 202 if the temperature is unsafe by comparing the temperature detected by the temperature sensor to a threshold temperature value. The threshold temperature value may vary, but preferably represents a temperature that would endanger an individual if exceeded for a continuous period of time. The threshold temperature value may refer to a particular high temperature which when exceeded poses danger to individuals or animals in a vehicle, or a particular low temperature which poses danger when the interior temperature becomes lower than the low temperature value. The logic also causes the vehicle monitoring system to determine 203 if the level of a gas such as CO2 within the vehicle interior is unsafe for individuals by comparing the concentration of CO2 to a threshold gas value. The threshold gas value may vary, but preferably represents a concentration of CO2 that would endanger an individual if he or she were exposed for a continuous period of time.

If the temperature level is unsafe 202 due to the temperature either exceeding or dipping below the threshold value, the logic causes a sound sensor such as a microphone to check 204 for vehicle occupants by measuring for the presence of sound within the interior of the vehicle. Similar to the temperature and gas sensors, a sound level threshold value may be set for the sound sensor such that it is determined that the vehicle is likely occupied if the sound level in the vehicle exceeds the sound level threshold value. In alternative embodiments, other sensors may be utilized to detect the presence of a vehicle occupant such as motion sensors and pressure sensors. In order to further improve occupant detection capabilities and reduce the likelihood of a false positive detection, which may be caused by increased CO2 emissions in the vehicle due to fruit left in the vehicle, an ethylene sensor may be utilized in conjunction with the CO2, since ripening fruit emits both CO2 and ethylene.

If the sound level within the vehicle exceeds the sound level threshold value, an occupant is detected 206, 207. The logic causes the vehicle monitoring system to communicate with the vehicle and cause it to perform a corrective action 208, 209. The corrective action 208, 209 is one that alleviates the dangerous condition within the vehicle and provides increased safety for the occupant, whether the dangerous condition is an increased temperature, unsafe CO2 concentration, or a combination of both. In some embodiments of the invention, the corrective action 207, 208 includes lowering vehicle windows in order to reduce the interior temperature and allow fresh air in to reduce the CO2 levels. The corrective action 207, 208 may also include turning on the vehicle air conditioning to further cool the interior of the vehicle and draw in outside air with a lower CO2 concentration that of the air in the interior of the vehicle. If the temperature in the vehicle dips below a lower threshold temperature, the air conditioning may be utilized to provide warm air. In alternative embodiments of the invention, the corrective action 207, 208 may include sounding the vehicle horn or flashing one or more of the vehicle exterior lights. These corrective actions 207, 208 are intended to alert nearby individuals to the dangerous conditions for the vehicle occupant so that they may help or contact emergency personal.

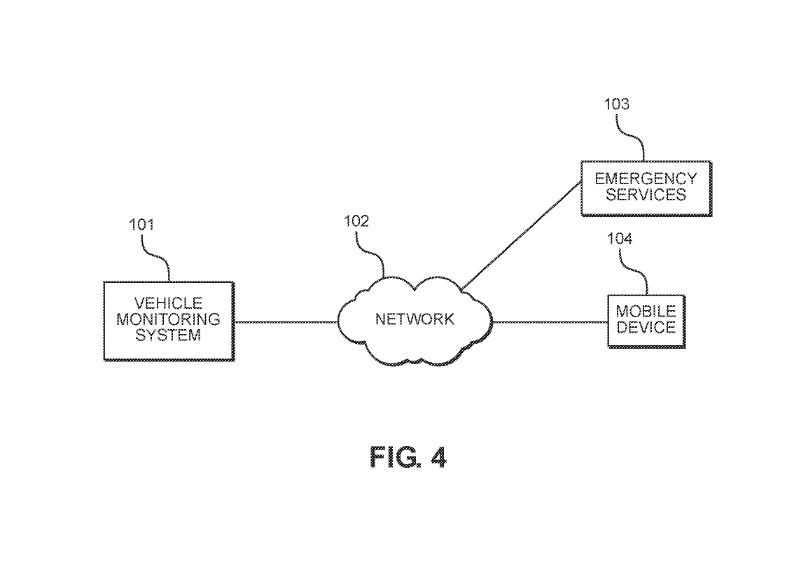

Referring now to FIG. 4, there is shown a schematic view of a vehicle monitoring system according to the present invention transmitting a signal to a third party. In addition to causing the vehicle to perform a corrective action, if a dangerous condition is present and the vehicle is occupied the vehicle monitoring system 101 transmits the location of the vehicle to a third party. In one embodiment, the location information is transmitted via a network 102 such as a cellular or Wi-Fi network to emergency services 103 such as the local police, fire department, or emergency dispatch center. The location information may also be transmitted to a mobile device 104 so that the user of the mobile device 104 may take additional action with regard to the dangerous condition. The user of the mobile device 104 may go to the vehicle location to alleviate the situation or may contact emergency services to report the condition or verify that the vehicle location has already been transmitted thereto.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A vehicle monitoring system comprising:
a processor;
a GPS;
a wireless transceiver;
a sound sensor;
a gas sensor;
a temperature sensor;
a non-transitory computer readable medium operatively connected to the processor;
a logic stored in the non-transitory computer readable medium that, when executed by the processor, causes the vehicle monitoring system to perform a method, the method comprising the steps of:
determining the ambient temperature in the interior of a vehicle,
if the ambient temperature in the interior of the vehicle exceeds a threshold temperature value, then determining the sound level in the interior of the vehicle,
if the sound level in the interior of the vehicle exceeds a threshold sound level, then causing the vehicle to perform a corrective action, and;
transmitting information to a third party over a network via the wireless transceiver upon the occurrence of the corrective action, wherein the information comprises the geographic location of the vehicle monitoring device as determined by the GPS.

2. The system of claim 1, wherein the method further comprises the steps of:
determining the concentration of CO2 in the interior of the vehicle,
if the concentration of CO2 in the interior of the vehicle exceeds a threshold concentration, then determining the sound level within the interior of the vehicle, and
if the sound level within the interior of the vehicle exceeds the threshold sound level, then causing the vehicle to perform the corrective action.

3. The system of claim 2, wherein the corrective action comprises lowering one or more windows of the vehicle.

4. The system of claim 2, wherein the corrective action comprises turning on an air conditioning unit of the vehicle, wherein the air conditioning unit is set at a temperature configured to lower the ambient temperature of the interior of the vehicle.

5. The system of claim 2, wherein the corrective action comprises sounding a horn of the vehicle.

6. The system of claim 2, wherein the corrective action comprises flashing one or more exterior lights of the vehicle.

7. The system of claim 1, wherein the vehicle monitoring system is in electrical and operable communication with the electrical systems and controls of a vehicle.

8. A non-transitory computer readable medium that, when executed by a processor of a vehicle monitoring system, causes the vehicle monitoring system to perform a method, the method comprising the steps of:
determining the ambient temperature in the interior of a vehicle,
if the ambient temperature in the interior of the vehicle exceeds a threshold temperature value, then determining the sound level in the interior of the vehicle,
if the sound level in the interior of the vehicle exceeds a threshold sound level, then causing the vehicle to perform a corrective action, and;
transmitting information to a third party over a network via the wireless transceiver upon the occurrence of the corrective action, wherein the information comprises the geographic location of the vehicle monitoring device as determined by the GPS.

9. The non-transitory computer readable medium of claim 8, wherein the method further comprises the steps of:
determining the concentration of CO2 in the interior of the vehicle,
if the concentration of CO2 in the interior of the vehicle exceeds a threshold value, then determining the sound level within the interior of the vehicle, and
if the sound level within the interior of the vehicle exceeds the threshold sound level, then causing the vehicle to perform the corrective action.

10. The non-transitory computer readable medium of claim 9, wherein the corrective action comprises lowering one or more windows of the vehicle.

11. The non-transitory computer readable medium of claim 9, wherein the corrective action comprises turning on an air conditioning unit of the vehicle, wherein the air conditioning unit is set at a temperature configured to lower the ambient temperature of the interior of the vehicle.

12. The non-transitory computer readable medium of claim 9, wherein the corrective action comprises sounding a horn of the vehicle.

13. The non-transitory computer readable medium of claim 9, wherein the corrective action comprises flashing one or more exterior lights of the vehicle.

14. The non-transitory computer readable medium of claim 9, wherein the vehicle monitoring system is in electrical and operable communication with the electrical systems and controls of a vehicle.
